(19) United States
(12) Patent Application Publication
(10) Pub. No.: US 2015/0368589 A1
(43) Pub. Date: Dec. 24, 2015

(54) AMPHOTERIC DETERGENT COMPATIBLE SOFTENERS BASED ON ALKYL AMMONIUM BACKBONE

(71) Applicant: Sandeepak PANDIT, Pune (IN)

(72) Inventor: Sandeepak PANDIT, Pune (IN)

(21) Appl. No.: 14/649,424

(22) PCT Filed: Jan. 28, 2013
(86) PCT No.: PCT/IN2013/000053

§ 371 Date: Jun. 3, 2015

(30) Foreign Application Priority Data
Dec. 6, 2012 (IN) 3445/MUM/2012

(51) Int. Cl. C11D 3/00; C07C 227/12; C07C 229/24; C11D 3/33; C11D 1/00

(52) U.S. Cl. CPC C11D 3/001; C11D 3/33; C11D 1/008; C07C 229/24; C07C 227/12

(57) ABSTRACT

The present invention provided amphoteric detergent compatible amphoteric softeners based on alkyl ammonium backbone of formula 1, 2 or 3. These softeners may be used directly to impart softness to the substrates such as fabrics. The invention also provides a method for preparation of these softeners by reaction of maleic anhydride with a mixture of long chain alcohols or glycols and then with a mixture of amine and olefinic acid and a polymerization inhibitor. The softeners of the present invention are used as such or in combination with cationic as well as anionic additives without any special additive. These softeners do not impart additional yellowing characteristics to the fabric.

### FIELD OF INVENTION

The present invention relates to detergent compatible softeners based on alkyl ammonium backbone. More particularly it relates to the said amphoteric softeners based on alkyl ammonium backbone structures as shown formulae 1 to 3 hereinbelow. Still more particularly it relates to the said softeners having in FORMULA-1 hereinbelow wherein R1 and R2 may be both H when R3 and R4 are nil

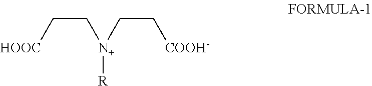

Or having formula 2 hereinbelow

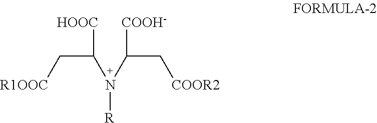

Wherein when R3 and R4 may be COOH, when R1=R2 and may be C12 to C22, saturated, or one double bond or two double bonds or may be polyethylene glycol or polypropylene glycol of molecular weights between 200 to 10000.

Or having formula 3 hereinbelow

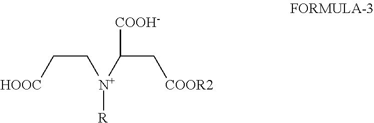

Wherein when R1=H, R3 is nil, R4=COOH and R2 may be C12 to C22, saturated, or one double bond or two double bonds and may be polyethylene glycol or polypropylene glycol of molecular weight between 200 to 10000.

Wherein in all the above formulae 1, 2 and 3, R=C12 to C22, saturated, one double bond or two double bonds or may be polyethylene glycol or polypropylene glycol of molecular weight between 200 to 10000.

The present invention also provides a process for the preparation of amphoteric the detergent compatible softeners by a novel route having formulae hereinabove as described in figures hereinabove.

The Invention also provides for the detergent compositions using the said softeners as mentioned hereinabove and its applications.

The detergents are used in solid or liquid forms for cleaning the fabrics since ancient times. These detergents when used also impart roughness to the fabrics or substrates on which they are used. To overcome this problem, the detergents are used in combination of additives i.e. compatible softeners which imparts softness to the substrates. Normally the detergents are used for washing and rinsing of substrates and subsequently softeners are used to have softening action on the substrates. The amphoteric detergent softeners have ability to give the softening effect to the substrates in presence of detergent. Optionally the softener is used in combination of scents, perfumes or similar agents for imparting fragrance and freshness to the substrates along with the softening effect of the softeners.

The compounds of formula 1, 2 and 3 or mixture thereof, provided by the present invention may be used as detergent softeners as such or may be used in combination of detergents.

The prior art has innumerable examples of compatible additives used in various kinds of detergents. In the closest prior art U.S. Pat. No. 6,346,648 to O'Lenick, Anthony J., Jr. teaches amphoteric surfactants base on epoxy succinic acid having formulae as given herein below:

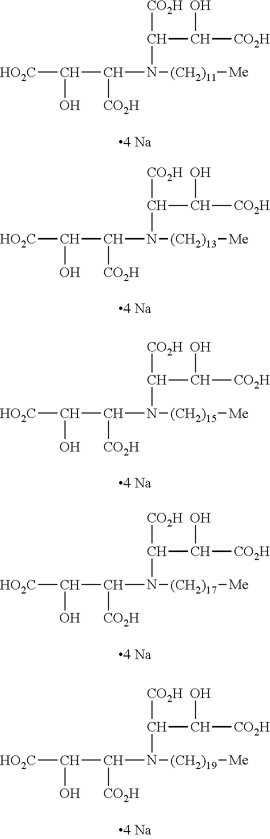

This patent describes amphoteric surfactants that contain a hydroxyl group and two carboxylic groups per hydroxyl group. The compounds have a unique structure, having multiple carboxyl groups on each amino group. The utility for these novel polymers is for softening, anti-tangle, and conditioning agents for use in personal care, textile and related applications. The process for preparation is in two steps as under.

Step A: Reacting maleic acid and sodium hydroxide to give disodium epoxy succinate

Step B: Reacting disodium epoxy succinate with tertiary amine to give amphoteric surfactants that contain a hydroxyl group and two carboxylic groups per hydroxyl group.

In another close prior art the Chinese patent titled “Alkyl iminodisuccinate salt chelating surfactant and its preparation method” to Zhengzhou Institute of Light Industry, People. Rep. China describes Alkylimino disuccinate chelate surfactant which is prepared by preparing a reactant mixture containing C8-18 fatty primary amines, maleic anhydride, and a base (e.g., NaOH) at mol ratio=1:(2-4):(4-8), adding a catalyst (e.g., copper chloride), and reacting at 80-180 deg C. and 0.1-5 MPa for 5-20 hr. The surfactant has both lipophilicity and hydrophilicity, good surface activity, and chelating ability.

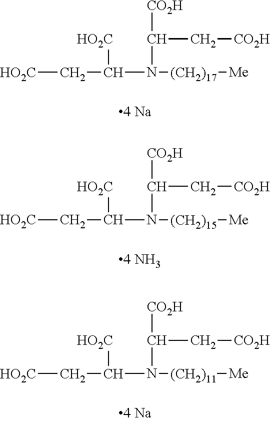

The surfactant hereinabove is prepared by reacting primary aliphatic amine and maleic anhydride, and synthesizing the alkylimino disuccinic acid or disuccinate using a one-step method. Preferred Components: The mole ratio of aliphatic amine:maleic anhydride:alkali is 1:2-4:4-8. A catalyst is added to react with the materials for 5-20 hours at 80-180 degrees C. and 0.1-5 MPa.

The present invention provided preparation of the detergent compatible softeners of formulae 1, 2 and 3 by a novel method described hereinafter for which no prior art is noticed by the inventor of the present invention.

The main object of the present invention is to provide amphoteric detergent compatible softeners.

Another object is to provide a process for the preparation of the said detergent compatible softeners wherein they are prepared by a novel route using maleic anhydride and fatty acid alcohol in the first step followed by reacting the intermediate product with stearyl amine and acrylic acid.

Still another object is to provide compositions of detergent using the said detergent compatible softeners.

### SUMMARY OF THE INVENTION

The present invention provides detergent compatible softeners based on alkyl ammonium backbone having formulae 1, 2 and 3 as provided hereinabove, a process for their preparation, the detergent compatible softener compositions using the said softeners and the application of these compositions to treat the substrates such as fabrics to secure softness.

The invention provides preparation of these detergent compatible softeners by a novel method for which no prior art is noticed.

### DETAILED DESCRIPTION OF THE INVENTION

Accordingly the present invention provides detergent compatible amphoteric softeners based on alkyl ammonium of hereinbelow wherein R1 and R2 may be both H when R3 and R4 are nil

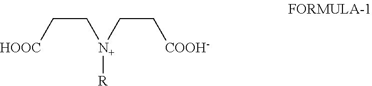

Or having formula 2 hereinbelow

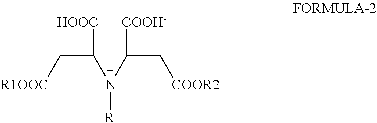

Wherein when R3 and R4 may be COOH, when R1=R2 and may be C12 to C22, saturated, or one double bond or two double bonds or may be polyethylene glycol or polypropylene glycol of molecular weight between 200 to 10000.

Or having formula 3 hereinbelow

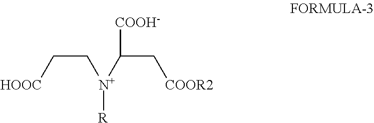

Wherein when R1=H, R3 is nil, R4=COOH and R2 may be C12 to C22, saturated, or one double bond or two double bonds or may be poly ethylene glycol or poly propylene glycol of molecular weight between 200 to 10000.

Wherein in all the above formulae 1, 2 and 3, R=C12 to C22, saturated, one double bond or two double bonds or may be polyethylene gycol or polypropylene glycol of molecular weight between 200 to 10000.

The present invention also provides a process for the preparation of detergent compatible amphoteric softeners based on alkyl ammonium backbone of formula 1, 2 or 3 as mentioned hereinbefore, which comprises
a) heating in an inert atmosphere, maleic anhydride, with a mixture of long chain alcohols or long chain glycols at 70-80° C. for ˜4 hrs.,
b) in a separate container adding to a mixture of an amine or mixture of amines or olefinic acid and reaction mixture obtained from step (a), heating the reaction mixture to a temperature of 60-65° C. for ˜2 hrs., adding to this a polymerization inhibitor, raising the temperature of reaction mixture to 80-120° C. within half an hour and further maintaining at the said temperature for 8-16 hrs., adjusting the pH of the reaction mixture with an alkali to 7 to 8 to obtain the product.

In one of the embodiments of the present invention the of long chain alcohols may be fatty alcohols exemplified by stearyl alcohol, oleyl alcohol, behenyl alcohol, synthetic alcohols exemplified by oxo alcohols or low molecular weight hydroxyl terminated polyesters or low molecular weight hydroxyl terminated polyethers.

In yet another embodiment the long chain glycols may be polyethylene glycol with molecular weight between 200 to 10000 or polypropylene glycol with molecular weight between 200 to 10000.

In still another embodiment the amine may be fatty amine, exemplified by stearyl amine, oleyl amine, behenyl amine or synthetic amine prepared by reacting fatty acid or synthetic acid with diethylene triamine (DETA) or triethylene tetra amine (TETA)

In yet another embodiment the olefinic acid may be acrylic acid, metha acrylic acid or itaconic acid.

In yet another embodiment the polymerization inhibitor exemplified by but not limited to catechol, monoethyl hydroquinone (MEHQ) or 4-turtiary butyl catachol

The present invention also provides a composition of the detergent softeners as claimed in claim 1 of formulae 1, 2, or 3 or mixture thereof wherein the composition comprises the compound of formulae 1, 2, 3 or mixture thereof, a detergent, an emulsifier and a fragrance imparting agent.

In another embodiment the concentration of the detergent in the composition may be is 0 to 98%

In yet another embodiment the emulsifier may be but not limited to polyoxyethylene alkyl aryl ether, alkyl phenol polyethanoxy ether or lauryl alcohol ethoxylates

In still another embodiment the concentration of the emulsifier may be 0.1 to 10.0%

### EXAMPLES

The following examples describe in details the process of preparation of the softener of the present invention, the compositions using the softeners of the present invention and also the applications of the said compositions in The examples are only illustrative and should not be construed to limit the scope of the present invention in any manner whatsoever.

### Example 1

### Step 1

In a four necked flask having stirrer and thermowell was nitrogen purged. In the said flask was taken 245 gm. Maleic Anhydride, 284 gm. Stearyl alcohol, 282 gm. Oleyl alcohol, 170 gm. Behenyl alcohol and the mixture was heated to 75° C. under slow stirring till the mixture becomes homogeneous. The stirring speed was raised to 80-90 rpm. And the temperature of the mixture was maintained between 70-80° C. for 4 hours. The acid value was checked and the mixture was allowed to cool to 40° C.

### Step 2

In a four necked flask well equipped with thermowell, stirrer, nitrogen purge, heating mantle 246 gms. of stearyl amine, 86 gms. of acrylic acid, 392 gms. of reaction mixture from step 1 and 100 gms. of Isopropy alcohol/Butyl cellosolve mixture were taken. The mixture was stirred at a constant speed of about 40-45 rpm. The mixture was heated slowly within 1 hour to 60-65° C., maintained the mixture at 60-65° C. for two hours and then added 3.5 gm. Monoethyl hydroquinone (MEHQ) to this mixture. The reaction temperature was raised to 85° C. within 30 minutes and maintained at 85° C. for 16 hrs. The pH was adjusted 7.5 using 50% NaOH/KOH mixture and then 124 gms Emulsifier OK 32 was added to the mixture and the reaction mixture was cooled to 40° C. to obtain the product

### Example 2

### Step 1

In a four necked nitrogen purged flask having stirrer and thermowell a mixture of 245 gms. of Maleic Anhydride 539.6 gm Stearyl alcohol, 98.06 gms. of Behenyl alcohol and 100 gm of Polypropylene glycol 2000 was heated to 76° C. slowly till the mixture becomes homogeneous.

To this mixture was added slowly within 30 minutes 200 gm of Polyethylene glycol 1000. Mixture was stirred at a speed of 80-90 rpm and held at 70-80° C. for 4 hours. Acid value of the mixture was checked and the reaction mixture was cooled to 40° C.

### Step 2

In another four necked flask well equipped with thermowell, stirrer and nitrogen purged, heating mentle was taken a mixture of 103.2 gm Diethylene teteramine (DETA) and 25 ml Toluene. The reaction mixture was heated under nitrogen at 75° C. and water of reaction was removed azeotropically using toluene azeotrope till the amine value of 330-350 is reached.

### Step 3

In another well equipped container equipped with thermowell, stirrer, nitrogen purge, heating mantle a mixture of 83.16 gm reaction mixture from STEP (2), 225 gm Olyl amine, 86 gm acrylic acid, 473.8 gm of reaction mixture from STEP 1 and 130 gm of Isp propyl alcohol (IPA) was taken and stirred at a speed of 40-45 rpm to obtain homogenous mixture. The stirring was continued for 12 hrs checking the stirring rpm after every 1-2 hours. This mixture was then heated slowly within 1 hour to 60-65° C. and maintained for two hours. To this mixture was added 3.5 gm

Monoethyl hydroquinone (MEHQ) and the temperature of the reaction mixture was increased to ˜90° C. within 30 minutes. The stirring speed was maintained at 40-45 rpm. And the reaction mixture was maintained at ˜90° C. for eight hours. The acid value of the reaction mixture was checked and maintained at 40-45. The reaction mixture was cooled to 40° C. The pH of the mixture was adjusted to 7.5 using 50% NaOH/KOH mixture. To this was added 100 gms of Noien DKX 405 and the solid content was adjusted to 30% using water.

### Example 3

### Step 1

In a four necked flask having stirrer, nitrogen purge and thermowell was taken a take a mixture of 245 gm Maleic Anhydride, 511.2 gm Stearyl alcohol, 163.3 gm Behenyl alcohol and the mixture was slowly heated to ˜75° C. to obtain homogeneous mixture. To this was added slowly within 30 minutes 200 gm of Polyethylene glycol 1000. Started to stir the mixture was maintained at ˜75° C. for 4 hours. The acid value of the reaction mixture was confirmed to 120 and the mixture was cooled to 40° C.

### Step 2

In another four necked flask well equipped with thermo well, stirrer, nitrogen purge, heating mantle a mixture of 246 gm stearyl amine, 86 gm acrylic acid, 448.2 gm of reaction mixture obtained in step (1) and 140 gm of IPA was taken. The mixture was stirred at ˜45 rpm. For 16 hrs keeping a check on rpm after every 1-2 hours. The reaction mixture was heated slowly to 65° C. within 1 hour and maintained at that temperature for two hours. 3.5 gm of monoethyl hydroquinone (MEHQ) was added to the mixture and the temperature of the mixture was raised to ˜120° C. within 30 minutes. The reaction mixture was maintained at ˜120° C. for 16 hours under constant stirring. The acid value was confirmed to be 120 and then the reaction mixture was cooled to 40° C. The pH was adjusef to 7-4 using 50% NaOH/KOH mixture. Thereafter 124 gm of Noigen Dainol 25 P was added to this mixture and total solid contents were adjusted to 30%

### Example 4

### Step 1

In a four necked flask having stirrer, nitrogen purge and thermowell a mixture of 245 gm Maleic Anhydride, 539.6 gm Stearyl alcohol, 98.06 gm of Behenyl alcohol 100 gm Polypropylene glycol 2000 was taken. The reaction mixture was heated to 80° C. slowly till the mixture becomes homogeneous. To this mixture 200 gm of Polyethylene glycol 1000 was added slowly within 30 days and the stirring started with a speed of 80-90 rpm. Keep the mixture at temperature between 80° C. for 4 hours. The acid value was confirmed to be 120 and the mixture was cooled to 40° C.

### Step 2

In another four necked flask well equipped with thermo well, stirrer, nitrogen purge, heating mantle etc. was taken a mixture of 246 gm stearyl amine, 86 gm acrylic acid, 473.8 gm of reaction mixture from step (1) and 142 gm IPA. Started stirring the mixture at a speed of 40-45 rpm and rpm was checked after every 1 hour. The mixture was stirred for 2 hrs and then heated slowly within 1 hour to ˜65° C. and maintained for two hours. Thereafter to this mixture 3.5 gm Monoethyl hydroquinone (MEHQ) was added and the temperature was increased to ˜125° C. within 30 minutes. The mixture was held at ˜125° C. for 16 hrs under constant stirring. The pH of the mixture was confirmed to be 2 to 3 (pH of 10% mixture in distilled water) and the mixture was cooled to ˜40° C. pH of the reaction mixture was adjusted to 7.8 using triethanol amine and 100 gm of Noigen DKX 405 was added and solid contents were adjusted to 85% using IPA

### Example 5

### Step 1

In a four necked flask having stirrer, nitrogen purge and thermowell a mixture of 245 gm maleic Anhydride, 539.6 gm stearyl alcohol, 98.06 gm behenyl alcohol and 100 gm Polypropylene glycol 2000 was taken. The mixture was heated to ˜80° C. slowly till the mixture becomes homogeneous. To this mixture was added slowly 200 gm of Polyethylene glycol 1000 within 30 minutes. Started to stir the mixture at ˜80 rpm and the mixture was maintained at ˜80° C. for 4 hours. The acid value was checked to be 110-120 and the mixture was cooled to 40° C.

### Step 2

In another four necked flask well equipped with thermowell, stirrer, nitrogen purge, heating mantle etc. a mixture of 123 gm Olyl amine, 225 gm stearyl amine, 86 gm acrylic acid, 473.8 gm reaction mixture from step (1) and 142 gm of IPA was taken. Started stirring the reaction mixture at a speed of ˜45 rpm. Checked the rpm after every 1-2 hours. Heated the reaction slowly within 1 hour to and maintained at 60-65° C. for two hours. To this mixture was then added 3.5 gm of Monoethyl hydroquinone (MEHQ) and the mixture was heated to ˜95° C. within 30 minutes and the reaction mixture was cooled to 40° C. The pH was adjusted to 8.0 using triethanol amine. To this mixture was added 100 gm of Niogen DKX 405 and the solid contents were adjusted to 85% using monoethyl glycol.

### Example 6

Study was undertaken to assess the effect of the detergent compatible softener or mixture thereof of the present invention (the softener provided by the present invention or the mixture thereof is termed as Kelsoft in this example) and the softness to the fabric when added to detergents. The experiment focus was to test the behavior on liquid detergent. In comparison, tests were conducted in powder detergent, which already contains bentonite that gives softness characteristic to the fabric.

### Experimental Details of the Study

Five tests were performed in the powder detergent OMO Multiação and in the liquid detergent Tixan Ypê. The amount of detergent used was based on the information contained in the package of each product. OMO Multiação indicates the use of 4/5 of a 200 ml glass for 35 L of water and Tixan Ypê indicates the use of a lid of the product for 10 L of water. For all tests it was used 5 L of water. See in Table 1 hereinbelow the graphic with the description of each test in the powder and liquid detergent In Table 2.

TABLE 1

| Test | Powder detergent | Kelsoft | Bentonite | Towels |
|---|---|---|---|---|
| 1 | 11.7 g | 0% | 0% | C |
| 2 | | 2.5% | 0% | B |
| 3 | | 5.0% | 0% | A |
| 4 | | 10.0% | 0% | D |
| 5 | | 0% | 4.0% | E |

TABLE 2

| Test | Liquid detergent | Kelsoft | Towels |
|---|---|---|---|
| 1 | 19.25 g | 0% | C |
| 2 | 19.25 g | 2.5% | B |
| 3 | 19.25 g | 5.0% | A |
| 4 | 19.25 g | 10.0% | D |
| 5 | 0 | 10.0% | E |

The tests described above underwent the following process:

In a recipient detergent, Kelsoft and/or others and 5 L of water were added. Then the towel was added and was stirred for two minutes in the said solution. The towel was allowed to stand for 30 minutes. The steps of stirring was repeated and the towel was allowed to rest for about 2 hrs. The assessment test was done after drying the towel.

Thereafter the samples underwent humidity absorption assessment test of Kelsoft resin.

Below is the process flowchart

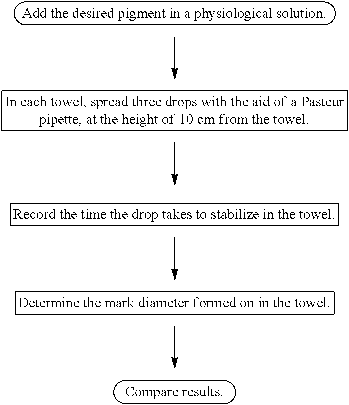

### Results and Observations

Softness assessment test:

Softness assessment results were made through research with twenty people who had to evaluate which were the softer towel and the rougher one. For liquid detergent the results are depicted in for softness in Graph 1 and roughness in Graph-2

Regarding softness it was observed that towel D, that corresponds to test 4 (detergent+10.0% Kelsoft), was evaluated as the softer towel, followed by towel E, that corresponds to test 5 (10.0% Kelsoft). These two towels often let people in doubt about which would be the softer one. Now regarding roughness, towel C, that corresponds to test 1 (detergent), was chosen for the most part, followed by towels B, that corresponds to test 2 (detergent+2.5% Kelsoft) and A, that corresponds to test 3 (detergent+5.0% Kelsoft).
For powder detergent:

Regarding softness it was observed that towel D, that corresponds to test 4 (detergent+10.0% Kelsoft), was evaluated as the softer towel, followed by towel E, that corresponds to test 5 (detergent+4.0% Argel 40). Regarding roughness, towel C, that corresponds to test 1 (detergent), was chosen as the rougher, followed by towel B, that corresponds to test 2 (detergent+2.5% Kelsoft).

### CONCLUSIONS

1. According to the results, resin Kelsoft gives the fabric the appearance of softness;

2. According to the tests, resin Kelsoft provided increase of humidity absorption capacity in the fabric, increasing up to 12.60% its absorption capacity in relation to a towel without resin;

3. When the resin Kelsoft concentration in the detergent is equal to or higher than 5.0% the distinction of softness is greater than in lower concentrations;

4. Kelsoft resin in combination with liquid and powder detergent provides some softness and enhances the humidity absorption.

### Advantages of the Invention

1. The present invention provides compounds of formulae provided herein before which are with Alkyl Ammonium Backbone as compared to quaternary ammonium based compounds.

2. The compounds provided by the present invention may be used as fabric softeners either as such or in combination of other detergents in contrast to other cationic or anionic detergent softeners.

3. Due to better humidity absorption by the fabrics, the product specially incorporates higher sweat absorption in fabrics.

4. The product can be combined with cationic as well as anionic additives without any special additive.

5. The product does not impart additional yellowing characteristics to the fabric.

I claim:

1. Detergent compatible amphoteric softeners based on alkyl ammonium of hereinbelow as shown in formula 1 wherein R1 and R2 are be both H when R3 and R4 are nil 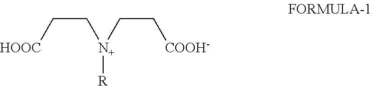
Or having formula 2 hereinbelow 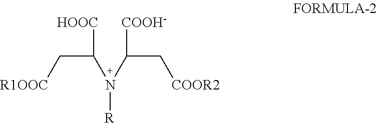
Wherein when R3 and R4 may be COON, when R1=R2 and and is C12 to C22, saturated, or one double bond or two double bonds or polyethylene glycol or polypropylene glycol of molecular weight between 200 to 10000.
Or having formula 3 hereinbelow 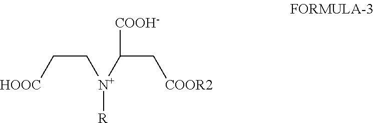
Wherein when R1=H, R3 is nil, R4=COOH and R2 is C12 to C22, saturated, or one double bond or two double bonds or poly ethylene glycol or poly propylene glycol of molecular weights between 200 to 10000.
Wherein in all the above formulae 1, 2 and 3, R=C12 to C22, saturated, one double bond or two double bonds or polyethylene glycol or polypropylene glycol.

2. A process for the preparation of detergent compatible amphoteric softeners based on alkyl ammonium backbone of formula 1, 2 or 3 as mentioned hereinbefore, which comprises
a) heating in an inert atmosphere, maleic anhydride, with a mixture of long chain alcohols or long chain glycols at 70-80° C. for ˜4 hrs.,
b) in a separate container adding to a mixture of an amine or mixture of amines or olefinic acid and reaction mixture obtained from step (a), heating the reaction mixture to a temperature of 60-65° C. for ˜2 hrs., adding to this a polymerization inhibitor, raising the temperature of reaction mixture to 80-120° C. within half an hour and further maintaining at the said temperature for 8-16 hrs., adjusting the pH of the reaction mixture with an alkali to 7 to 8 to obtain the product.

3. A process as claimed in claim 2 wherein the long chain alcohols are fatty alcohols exemplified by stearyl alcohol, oleyl alcohol, behenyl alcohol, synthetic alcohols exemplified by oxo alcohols or low molecular weight hydroxyl terminated polyesters or low molecular weight hydroxyl terminated polyethers.

4. A process as claimed in claim 2 wherein the long chain glycols are polyethylene glycol of molecular weight between 200 to 10000 or polypropylene glycol of molecular weight between 200 to 10000.

5. A process as claimed in claim 2 wherein the amine is fatty amine, exemplified by stearyl amine, oleyl amine, behenyl amine or synthetic amine prepared by reacting fatty acid or synthetic acid with diethylene triamine (DETA) or triethylene tetra amine (TETA).

6. A process as claimed in claims 2 wherein the olefinic acid is acrylic acid, metha acrylic acid or itaconic acid.

7. A process as claimed in claims 2 wherein the polymerization inhibitor is exemplified by but not limited to catechol, monoethyl hydroquinone (MEHQ) or 4-turtiary butyl catechol.

8. A composition of the detergent softeners as claimed in claim 1 of formulae 1, 2, or 3 or mixture thereof wherein the composition comprises the compound of formulae 1, 2, 3 or mixture thereof, a detergent, an emulsifier and a fragrance imparting agent.

9. A composition as claimed in claim 8 wherein the concentration of the detergent in the composition is 0 to 98%

10. A composition as claimed in claim 8 wherein the emulsifier is but not limited to polyoxyethylene alkyl aryl ether, alkyl phenol polyethanoxy ether or lauryl alcohol ethoxylates.
